(19) United States
(12) Patent Application Publication
(10) Pub. No.: US 2021/0016054 A1
(43) Pub. Date: Jan. 21, 2021

(54) SYSTEM AND METHOD OF REDUCING OR ELIMINATING ANXIETY

(71) Applicant: Bruce Gottlieb, St. George, UT (US)

(72) Inventor: Bruce Gottlieb, St. George, UT (US)

(21) Appl. No.: 16/933,787

(22) Filed: Jul. 20, 2020

Related U.S. Application Data
(60) Provisional application No. 62/876,451, filed on Jul. 19, 2019.

(51) Int. Cl. A61M 21/02; G06F 3/01

(52) U.S. Cl. CPC A61M 21/02; A61M 2021/0027; G06F 3/015

(57) ABSTRACT

A system and method of reducing or eliminating anxiety has a display device, an anxiety producing stimuli, a biofeedback system, and relaxation therapy. The display device displays the anxiety producing stimuli for a client. When the client becomes stimulated due to the anxiety producing stimuli, the biofeedback systems alerts the client that their heart rate is increasing. Once the heart rate increases, the client will go through relaxation therapy until the client reaches a relaxed state. The client is then presented with the same anxiety producing stimuli. This is repeated until the client can endure the stimulation without an increase in anxiety.

### CROSS-REFERENCE TO RELATED APPLICATIONS

This application claims the benefit of U.S. Provisional Application Ser. No. 62/876,451, filed on Jul. 19, 2019, which is incorporated herein by reference.

### TECHNICAL FIELD

The present disclosure relates to a system and method for reducing or eliminating anxiety. More particularly, the present disclosure relates to a system and method of reducing or eliminating anxiety by combining virtual reality and electronic bio-feedback with a titrated therapy protocol.

### BACKGROUND

Human beings have had to find ways to deal with anxiety since the beginning of time. Many of the rudimentary ways of dealing with anxiety have unending deficiencies. While some clients may use simple ways to deal with anxiety, others may not even know how to deal with its effects. Overall, therapists have found that no cures exist for psychiatric responses, such as anxiety; however, therapists have found techniques to shift psychological patterns from hijacking emotions. Therapists have also sought many approaches to help clients deal with anxiety in many forms, such as through breathing techniques or medication. Often, these approaches focus on either the physiological or psychological components within a client.

With this approach, the anxiety shifting techniques are not as effective, because focusing on physiological or psychological components separately will produce a relatively short beneficial effect due to the fact that a client's distress only partially lies in either the physiological or psychological. When therapists help clients make shifts in their behaviors through purely psychological means, the treatment will only address a portion of the client's issues. For example, pharmacology, although it serves a function, typically is a symptom suppressor and does not address the underlying problem. Over the past years many new treatments that are more related to the body have appeared. However, these treatments are still lacking effectiveness when it comes to assisting a client address the underlying issues.

Understanding the autonomic nervous system (ANS) is essential in helping a client address anxiety. The ANS consists of the sympathetic branch and the parasympathetic branch. The sympathetic branch controls what is commonly known as “the fight or flight” response, while the parasympathetic branch relaxes a client. Monitoring both of these branches allows a therapist to assist a client. Curtailing the effects of the sympathetic branch is a major part of helping someone with their anxiety. In addition to analyzing both branches of the ANS, a therapist must look at the physiological and psychological components together. Therapists have tried to approach the physiological and psychological components in a client through exposure therapy with varying results.

Exposure therapy, used in conjunction with virtual reality devices, takes a client's fear and exposes the client to the fear until the client professes to no longer be afraid. Although the client might exhibit fewer symptoms of anxiety immediately following exposure therapeutic techniques, the long-term effect of heightening a person's anxiety through artificial means does not help the client integrate these short-term positive effects. The best approaches to helping clients achieve long-term, positive effects from therapy should address the physiological and the psychological components and how they interact, because looking at one will only give a partial answer.

Accordingly, there is a need for a system and method of reducing or eliminating anxiety that can be self-administered, utilizes virtual reality, has electronic bio-feedback which allows a client to gain control of the therapy, and that addresses the physiological and psychological components of a client simultaneously. The present disclosure seeks to solve these and other problems.

### SUMMARY OF EXAMPLE EMBODIMENTS

In one embodiment, a system and method of reducing or eliminating anxiety comprises a display device for presenting an anxiety producing stimuli, a biofeedback system, and relaxation therapy. The display device displays the anxiety producing stimuli for a client. When the client becomes stimulated due to the anxiety producing stimuli, the biofeedback systems tells the client that their heart rate is increasing. Once the heart rate increases, the client will go through relaxation therapy until the client reaches a relaxed state. The client is then again presented with the same anxiety producing stimuli and the process repeats until the client's heart rate does not increase above a predetermined threshold when presented with the anxiety producing stimuli.

In one embodiment, a system and method of reducing or eliminating anxiety comprises a virtual reality device as a display device.

In one embodiment, a system and method of reducing or eliminating anxiety comprises a signaling device.

### BRIEF DESCRIPTION OF THE DRAWINGS

FIG. 1 illustrates a flow chart of a system and method of reducing or eliminating anxiety; and

FIG. 2 illustrates a flow chart of a system and method of reducing or eliminating anxiety.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As previously discussed, there is a need for a system and method of reducing or eliminating anxiety that can be self-administered, utilizes virtual reality, has electronic bio-feedback which allows a client to gain control of the therapy, and that addresses the physiological and psychological components of a client simultaneously. The present disclosure seeks to solve these and other problems.

Generally, the system and method of reducing or eliminating anxiety comprises a display device that may display an anxiety producing stimuli that incites the autonomic nervous system (ANS). The ANS is essential in addressing anxiety. The ANS consists of two branches: (1) the sympathetic branch and (2) the parasympathetic branch. The sympathetic branch controls what is commonly known as “the fight or flight” response, while the parasympathetic branch relaxes a client. The system of reducing or eliminating anxiety allows a therapist to assist a client in controlling the sympathetic branch and focus on activating the parasympathetic branch.

Further, the system focuses on the physiological and psychological components together to help clients achieve long-term, positive effects from therapy. While a client is using the system of reducing or eliminating anxiety, a biofeedback system monitors and displays to the client and the therapist the client's heartrate. The heartrate will be monitored so as to decrease the likelihood of overstimulating the client. To reduce the client's heartrate, by controlling their anxiety, relaxing images or sound is introduced when the client is becoming too anxious. It will be appreciated that this system allows a client to experience anxiety in a simulation setting and learn how to navigate their way through an anxious situation without being in the actual anxiety-producing situation.

In one embodiment, a system and method of reducing or eliminating anxiety comprises a display device for presenting an anxiety producing stimuli, a biofeedback system, and relaxation therapy. The display device (e.g., T.V., phone, tablet, virtual reality device, augmented reality device, etc.) is any device that displays the anxiety producing stimuli to a client. While the display device comprises a visual screen, audio may also be used. For example, a speaker may be used to offer anxiety producing stimuli in audio form (e.g., sound of infant crying, crowd screaming, etc.). Audio stimulus is also beneficial to help those that have anxiety issues and are visually impaired. The anxiety producing stimuli can vary and is specific to each client's anxiety. For example, one client may have anxiety attacks when driving on a freeway, while a different client may have anxiety attacks while boarding a plane. These situations are displayed to the client as a simulation and act as the anxiety producing stimuli.

The biofeedback system (i.e., heart rate monitor (e.g., smartwatch) and heart rate notification (e.g., visual number on tablet) combined) notifies the client of their heart rate and any increases. In one embodiment, when the client's heart rate rises 3-5 beats, the display device notifies the client visually or audibly of their increased heart rate. While a rise of 3-5 heartbeats may be the desired predetermined threshold, it can be appreciated that other ranges may be used, such as 2-6, 3-8, etc. At the time of the rise in heart rate, the client has the opportunity to pause the anxiety producing stimuli. If the client does not pause the anxiety producing stimuli, then the system of reducing or eliminating anxiety automatically pauses within the next few beat increases (e.g., 2, 4, 6) of the client's heart rate. This auto-pausing feature ensures that the client is not over-stimulated.

In contrast, exposure theory therapy pushes the client to over-stimulation, which does not produce beneficial long-term effects. Therefore, in the present system and method of reducing or eliminating anxiety, the client gains control of the stimuli and is not over-stimulated because the client can stop the anxiety producing stimuli or it will be stopped automatically. Without the ability to stop the anxiety producing stimuli, the brain moves the client into an anxious response due to a series of emitted chemicals. These chemicals shutdown the thinking process and cause anxiety in the client. In contrast, the system and method of reducing or eliminating anxiety slows the process and keeps the client within manageable, coherent parameters.

Once the anxiety producing stimuli has been paused, the system and method of reducing or eliminating anxiety moves into relaxation therapy. In one embodiment, the relaxing therapy comprises visual scenes, such as jellyfish smoothly moving through water, or a person sitting by a calm, beautiful lake in the mountains. In one embodiment, the relaxing therapy has a soft, peaceful voice instructing the client through breathing techniques during the visual scene. Conscious, slow breathing can stimulate the parasympathetic branch of the autonomic nervous system. This allows the client to learn how to relax before being overstimulated, which overstimulation can turn into an out-of-control sympathetic response. It will be appreciated that the relaxing therapy may be in a variety of forms such as images, voices, music, etc.

After control has been obtained and the client's heart rate has been at a normal rate for a predetermined period of time (e.g., 30 sec., 1 min., 5 mins., etc.), the system and method of reducing or eliminating anxiety returns to the anxiety producing stimuli. However, the anxiety producing stimuli does not resume at the prior paused location; rather, it goes back far enough that the client has the opportunity to learn how to prepare for the anxiety producing stimuli before it appears. The system and method of reducing or eliminating anxiety continues until the client can successfully move through the entire procedure without experiencing an increase in heart rate.

It will be appreciated that the system and method of reducing or eliminating anxiety uses anxiety producing stimuli in a titrated (continuously measured and adjusted) manner. This prevents overstimulation of the sympathetic branch of the autonomic nervous system. With a titrated anxiety producing stimuli, the client learns to identify the early signs of their anxiety and how to bring themselves into a regulated state. All of this helps the clients access interior and exterior resources, which helps them move to the more regulated state.

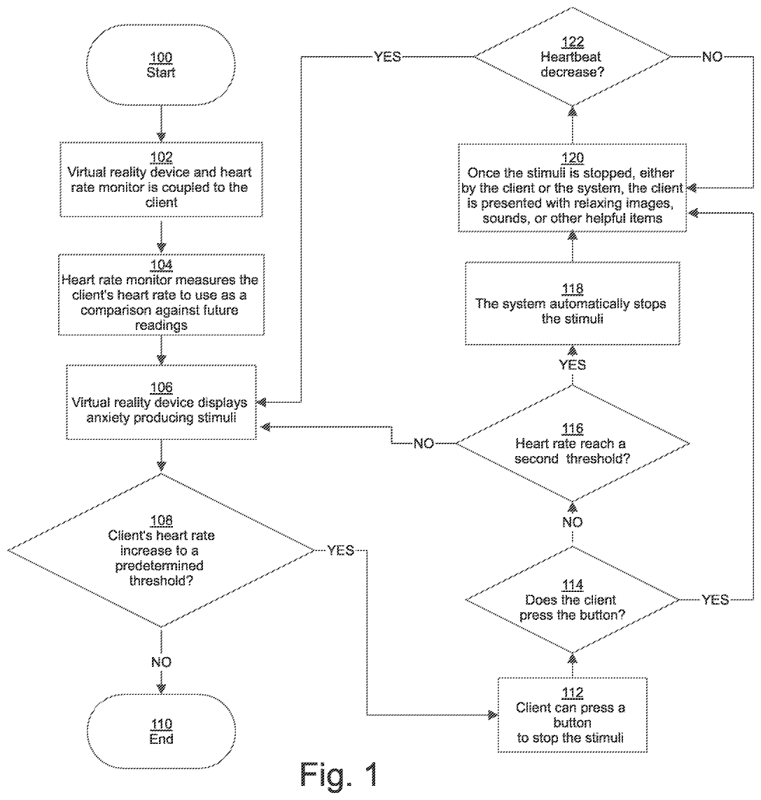

As illustrated in the flow chart in FIG. 1, the system starts 100. At step 102, the virtual reality device and heart rate monitor is coupled to the client. For example, the virtual reality device may be those known in the art, including those that utilize smartphones for the screen and those with screens built in. Further, the heart rate monitor may be in any known form, such as medical devices or smartwatches. At step 104, the heart rate monitor measures the client's heart rate to use as a comparison against future readings. At 106, anxiety producing stimuli is presented to the user via the virtual reality device. At step 108, the system determines whether there was an increase in heart rate. For example, the biofeedback device/system may include a processor or microcontroller for comparing heart rate data; in the alternative, a third-party device may be used, such as a computer, tablet, or smartphone, to effectuate the biofeedback. The tablet, heartrate monitor, and virtual reality device may all be wirelessly connected. If the client's heart rate did not increase, the system ends in step 110. Alternatively, the system may present additional or alternative stimuli until a heart rate response is achieved. Returning to the system, if the client's heart rate did increase to a predetermined threshold, the client can press a button, in step 112, to stop the stimuli. Again, this button may be connected to the microcontroller of the system, or may be coupled to the third-party device, if used. If the client does not stop the stimuli in step 114, and the client's heartrate continues to increase, as measured in step 116, the system will automatically stop (such as by using software) the stimuli at step 118. The client and/or therapist are made aware of the change in heart rate using the biofeedback system (i.e., heart rate monitor coupled with heart rate notification—such as a visual indicator on the screen or audible indicator). Once the anxiety producing stimuli is stopped, either by the client or by the software of the system, the client is presented with relaxing images, sounds, or other helpful items in step 120. In step 122, the system determines if the heartrate has decreased below the predetermined threshold. If the heartrate decreased, then the system returns to step 106. If the heartrate did not decrease, step 120 continues until the heartrate is reduced. It will be appreciated that this method allows a client to control their own anxiety by controlling the exposure of the anxiety inducing stimuli. This allows a client to feel comfortable during the process. If the client fails to pause the stimuli, and the therapist has set a predetermined threshold for safety and/or efficacy, the system will automatically pause to ensure the client is not overexposed. This is a major improvement over the prior art. Further, the therapist may have a tablet, computer, or other device that communicates with the virtual reality device and mirrors what the client is being shown, allowing the therapist to understand the client better and maintain control of circumstances and understand the client's heartrate increases. Duplicate views and control of virtual reality devices are well known in the art, all of which may be used herewith.

In one embodiment, the client knows their heartrate has increased using visual cues presented on the screen. In another embodiment, the client knows their heartrate has increased using audible tones. In yet another embodiment, both visual and audio cues are used to inform the client and/or the therapist of the heartrate. In one embodiment, the system and method of reducing or eliminating anxiety comprises a wireless transceiver (e.g., Wi-Fi®, Bluetooth®, etc.). This allows a therapist to wirelessly control/monitor the system, either from a user control interface (e.g., touchscreen, button interface, etc.) or a third-party wireless device, such as a computer, tablet, smartphone, etc.

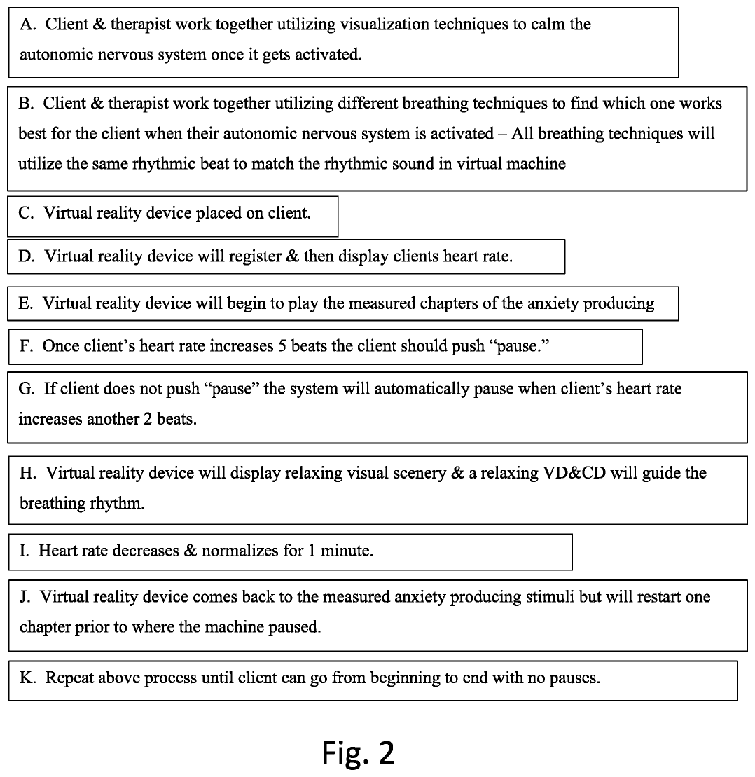

As shown in FIG. 2, a non-limiting example of a process of using the system and method of reducing or eliminating anxiety 200, in one embodiment, includes the following steps: A.) Client and therapist work together utilizing visualization techniques to calm the autonomic nervous system once it gets activated; B.) Client and therapist work together utilizing different breathing techniques to find which one works best for the client when their autonomic nervous system is activated—the breathing techniques may utilize the same rhythmic beat to match the rhythmic sound, if presented to the client C.) Virtual reality device placed on client. D.) Virtual reality device will register & then display client's heart rate from heart rate measuring device. E.) Virtual reality device will begin to play the measured chapters of the anxiety producing stimuli. F.) Once client's heart rate increases 5 beats the client should push “pause.” G.) If client does not push “pause” the system will automatically pause when client's heart rate increases another 2 beats (i.e., software executes to pause the stimuli). H.) Virtual reality device will display relaxing visual scenery & a relaxing voice will guide the breathing rhythm. I.) Heart rate decreases & normalizes for 1 minute. J.) Virtual reality device comes back to the measured anxiety producing stimuli, but will restart one chapter prior to where the system paused. K.) Repeat above process until client can go from beginning to end with no pauses.

In one embodiment, the system and method of reducing or eliminating anxiety comprises a virtual reality device as a display device. The virtual reality device displays an anxiety producing stimuli, such as an anxious situation. The virtual reality device allows a client to feel as if they are actually living through the anxiety-filled moment. The virtual reality device also displays relaxing visual scenes after the client has been anxiously stimulated to a predetermined threshold.

In one embodiment, a system and method of reducing or eliminating anxiety comprises a signaling device. The signaling device may comprise a wireless transceiver, such as a Bluetooth® device, but may also be other wireless or even wired devices, that registers a client's heart rate. The heart rate can be viewed on the display device or be an audible alert, such as a voice or beep. As the client views their heart rate or hears heartrate alerts, they can learn to monitor their anxiety when faced with anxiety producing stimuli.

It can be appreciated that the system and method of reducing or eliminating anxiety keeps the client in manageable parameters, rather than overexposing the brain to anxiety producing stimuli, like exposure therapy. Being able to titrate the anxiety producing stimuli allows a client to understand how to control and manage their anxiety. This is in stark contrast to the prior art's exposure therapy. Exposure therapy pushes the client to the breaking point, making them face their fears. There may be some beneficial short-term effects, but the long-term beneficial effects are lacking. In contrast, the system and method of reducing or eliminating anxiety disclosed herein is a softer approach that brings deeper and longer-lasting relief to the client, while at the same time educating them how to better control an anxious situation.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A system for reducing or eliminating anxiety, comprising:
a display device;
an anxiety producing stimuli presented to a user on the display device;
a biofeedback system to monitor the heart rate of the user and detect a raise in heart rate; and
relaxation therapy presented to the user on the display device when the heartrate increases to a predetermined threshold.

2. The system of claim 1, wherein the display device comprises a virtual reality device.

3. The system of claim 1, wherein the display device comprises a speaker.

4. The system of claim 1, wherein the predetermined threshold is a rise of 3-5 heartbeats.

5. The system of claim 1, wherein the predetermined threshold is a rise of 2-6 heartbeats.

6. The system of claim 1, wherein the relaxation therapy comprises a soft voice instructing the user through breathing techniques.

7. The system of claim 1, where the relaxation therapy comprises a visual nature scene.

8. A system for reducing or eliminating anxiety, comprising:
a virtual reality device;
an anxiety producing stimuli presented to a user on the virtual reality device;
a biofeedback system comprising a heart rate monitor and microcontroller to monitor the heart rate of the user and detect a raise in heart rate; and
when an increased heartrate is detected, presenting relaxation therapy to a user on the virtual reality device.

9. The system of claim 8, wherein the relaxation therapy comprises audio.

10. A method of reducing or eliminating anxiety using the system of reducing or eliminating anxiety, the method comprising:
placing a virtual reality device on a user;
displaying the user's heart rate and an anxiety producing stimuli on the virtual reality device;
monitoring the heart rate during the anxiety producing stimuli;
pausing the anxiety producing stimuli when the user's heart rate increases to a predetermined threshold;
displaying relaxation therapy until the user's heart rate decreases for a predetermined period of time;
restarting the anxiety producing stimuli;
wherein the user completes the process when confronting the anxiety producing stimuli fails to increase the heartrate.

11. The method of claim 10, wherein the display device comprises speaker.

12. The method of claim 10, wherein when the user's heart rate raises 3-5 heartbeats, the predetermined threshold is reached.

13. The method of claim 10, wherein the period of time for a decreased heartrate is 1 minute.

14. The system of claim 10, wherein the relaxation therapy comprises a soft voice instructing the user through breathing techniques.

15. The system of claim 10, where the relaxation therapy comprises a visual nature scene.
